(19) United States
(12) Patent Application Publication
(10) Pub. No.: US 2018/0263612 A1
(43) Pub. Date: Sep. 20, 2018

(54) SURGICAL RETRACTOR AND WIRE PASSER

(71) Applicants: C. Daniel Smith, St. Joseph, MO (US); Tadd Nicholas Smith, Rye, NH (US); Judd Michael Smith, Lebanon, NH (US)

(72) Inventors: C. Daniel Smith, St. Joseph, MO (US); Tadd Nicholas Smith, Rye, NH (US); Judd Michael Smith, Lebanon, NH (US)

(21) Appl. No.: 15/459,892

(22) Filed: Mar. 15, 2017

(51) Int. Cl. A61B 17/02

(52) U.S. Cl. CPC A61B 17/02

(57) ABSTRACT

A surgical retractor and wire passer broadly comprising an elongated handle, a paddle, a curved tip, and a guide passageway. The elongated handle allows a user to grasp and apply leverage to the surgical retractor and wire passer. The paddle extends from the elongated handle and is a thin and flat plate for holding back non-skeletal tissue away from an operating area. The curved tip extends from the paddle opposite the handle and includes a concave engagement surface for engaging the bone. The guide passageway extends along the curved tip and curls a surgical wire at least partially around the bone.

### BACKGROUND

Bone fractures such as subtrochanteric and periprosthetic fractures often require surgery in which a bone brace or splint is attached to two or more fractured bone segments via surgical wire. During surgery, surgical retractors are typically used for holding non-skeletal body mass such as arteries, muscles, and fat away from the operating area and surgical wire passers are typically used for guiding the surgical wire around the bone segments. However, this often requires an assistant to operate the surgical retractor while a surgeon or other personnel operates the wire passer. The surgical retractor operator and the wire passer operator often must coordinate their actions due to the limited access and congestion of the operating area. The surgical retractor operator and the wire passer operator also must anticipate each other's actions to perform the operation fluidly, which may result in unnecessary, inefficient, or incorrect surgical actions being taken.

### SUMMARY

Embodiments of the present invention solve the above-described problems and provide a distinct advance in surgical tools. An embodiment of the invention is a surgical retractor and wire passer broadly comprising an elongated handle, a paddle, a curved tip, and a guide passageway.

The elongated handle extends upward from the paddle and allows a user to grip and apply a leveraging force to the surgical retractor and wire passer. The elongated handle may be include ergonomic gripping contours, ridges, protrusions, or other geometry for allowing the user to firmly grasp the surgical retractor and wire passer.

The paddle is a thin and wide plate for holding non-skeletal body mass away from the operating area and has opposing front and back surfaces and opposing proximal and distal ends. The paddle is connected to or transitions to the handle at the proximal end and the curved tip at the distal end. The paddle may be angled relative to the elongated handle at its proximal end or between its proximal and distal ends.

The curved tip extends from the distal end of the paddle and includes a concave engagement surface for engaging the bone. The concave engagement surface may have one of a number of different curvature arcs and radii of curvature for engaging bones of different sizes.

The guide passageway guides a surgical wire at least partially around the bone and may be a closed-wall, open-ended channel; tube; groove; or other suitable geometry. The guide passageway may extend only along the curved tip or may extend along at least a portion of the paddle.

In use, the surgical wire retractor and wire passer may be anchored against the bone via the concave engagement surface of the curved tip. The elongated handle may then be pushed or pulled such that the back surface of the paddle engages the non-skeletal body mass and urges it away from the operating area.

The surgical wire may then be inserted into the guide passageway and pushed along the guide passageway towards the end of the curved tip. The guide passageway will curl the end of the surgical wire at least partially around the bone. The end of the surgical wire may then be received on the other side of the bone. The surgical wire may then be clipped near the paddle between 2 inches and 5 inches from the end of the surgical wire to form a wire piece. The wire may then be secured to a brace or other reinforcement.

Another embodiment is a surgical retractor and wire passer broadly comprising an elongated handle, a paddle, a curved tip, and two or more guide passageways. The elongated handle, paddle, and curved tip are substantially similar to the elongated handle, paddle, and curved tip described above. The guide passageways are substantially similar to the guide passageway described above except that the guide passageways are laterally spaced from each other. This allows two or more surgical wires to be passed around the bone at once or in succession.

### BRIEF DESCRIPTION OF THE DRAWINGS

FIG. 1 is a perspective view of a surgical retractor and wire passer constructed in accordance with an embodiment of the invention; and

FIG. 2 is a perspective view of a surgical retractor and wire passer constructed in accordance with another embodiment of the invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention may be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

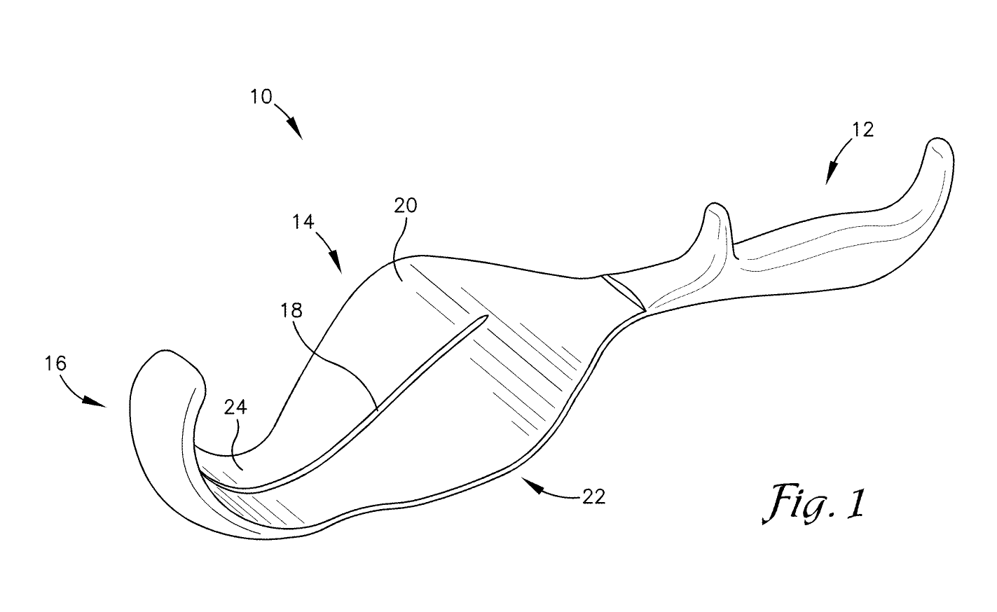

Turning now to FIG. 1, a surgical retractor and wire passer 10 is illustrated in accordance with an embodiment of the invention. The surgical retractor and wire passer 10 broadly comprises an elongated handle 12, a paddle 14, a curved tip 16, and a guide passageway 18.

The elongated handle 12 extends upward from the paddle 14 and allows a user to grip and apply a leveraging force to the surgical retractor and wire passer 10. The elongated handle 12 may include ergonomic gripping contours, ridges, protrusions, or other geometry for allowing the user to firmly grasp the surgical retractor and wire passer 10. The elongated handle 12 may have a thickness of between 0.5 inches and 2 inches and preferably between 0.5 inches and 1 inch. The elongated handle 12 may have a thickness greater than at least 1.5, preferably at least 2, and more preferably at least 3 times the thickness of the paddle 14.

The paddle 14 is a thin and wide plate for holding non-skeletal body mass away from the operating area and has opposing front and back surfaces 20, 22 and opposing proximal and distal ends. The paddle 14 extends downward from the elongated handle 12 at the proximal end of the paddle 14 and may be at least partially angled relative to the elongated handle 12. More specifically, the paddle 14 may be angled at its proximal end or may be angled between its proximal and distal ends. The angle may be between 170 degrees from parallel and 90 degrees and preferably between 160 degrees from parallel and 90 degrees. In this way, the paddle 14 may be substantially flat or may have a slight concave curvature or angle such that the back surface 22 can slightly cradle the non-skeletal body mass. The paddle 14 is connected to or transitions to the curved tip 16 at the distal end of the paddle 14. The paddle 14 may have a thickness of between 0.125 inches and 0.5 inches, preferably between 0.125 inches and 0.4 inches, and more preferably between 0.125 inches and 0.25 inches. The paddle 14 may have a width of between 2 inches and 5 inches and preferably between 2 inches and 4 inches. The width of the paddle 14 may be at least 2, preferably at least 4, and more preferably at least 6 times the widths of the elongated handle 12 and the curved tip 16.

The curved tip 16 extends from the distal end of the paddle 14 and includes a concave engagement surface 24 for engaging the bone. The concave engagement surface 24 may have a curvature arc of at least 20 degrees, preferably at least 60 degrees, and more preferably at least 90 degrees. The curvature arc may also be at most 180 degrees, preferably at most 160 degrees, and more preferably at most 140 degrees. The concave engagement surface 24 may have a radius of curvature of between 0.25 inches and 2 inches, between 0.5 inches and 4 inches, between 0.75 inches and 3 inches, or between 1 inch and 2 inches for engaging bones of different sizes.

The guide passageway 18 guides a surgical wire at least partially around the bone and may be a closed-wall, open-ended channel; tube; groove; or other suitable geometry. The guide passageway 18 may extend only along the curved tip 16 or may extend along at least a portion of the paddle 14. The guide passageway 18 may have a diameter or width of at least 0.125 inches and preferably at least 0.25 inches for receiving surgical wires of different sizes.

Use of the surgical retractor and wire passer 10 will now be described in more detail. First, a user may grasp the elongated handle 12 and insert the curved tip 16 into the operating area near a bone that needs to be braced or reinforced. The concave engagement surface 24 of the curved tip 16 may then be positioned against the bone such that the curved tip 16 cradles the bone. The surgical retractor and wire passer 10 may need to be angled forward briefly to allow the end of the curved tip 16 to clear the bone.

The back surface 22 of the paddle 14 may then be pushed or pulled against the non-skeletal body mass with the curved tip 16 anchored against the bone so as to hold the non-skeletal body mass away from the operating area. The surgical retractor and wire passer 10 will thus generate leverage, making the non-skeletal body mass easier to hold away from the operating area.

The surgical wire may then be inserted into the guide passageway 18. The surgical wire may then be pushed along the guide passageway 18 towards the end of the curved tip 16 so that the guide passageway 18 curls the end of the surgical wire at least partially around the bone. The end of the surgical wire may then be received on the other side of the bone and the surgical wire may be clipped near the paddle 14 between 2 inches and 5 inches from the end of the surgical wire to form a wire piece at least partially encircling the bone. The wire piece may then be secured to a brace or other reinforcement near both ends of the wire piece.

The surgical retractor and wire passer 10 may then be disengaged from the wire piece (i.e., the wire piece may be removed from the guide passageway 18). The surgical retractor and wire passer 10 may then be moved laterally (axially along the bone) for passing another surgical wire around the bone. This may be repeated until the brace is completely secured to the bone.

The curved tip 16 may then be removed from the bone. In doing so, the paddle 14 may release tension on the non-skeletal body mass as the paddle 14 is angled forward. Alternatively, the user may simultaneously retain pressure against the non-skeletal body mass via the paddle 14 while disengaging the curved tip 16 from the bone.

The above-described surgical retractor and wire passer 10 provides several advantages over conventional devices. For example, the surgical retractor and wire passer 10 holds non-skeletal body mass away from the operating area while simultaneously guiding the surgical wire around the bone. This eliminates the need for additional tools to complete this task. Fewer tools eliminates the reliance on an assistant and/or puts less of a burden on the user, which reduces the cost and time requirement of the procedure. Fewer tools also results in a less cluttered operating area, which makes the surgery easier to perform. A user can easily engage the handle with one hand to move and hold back the non-skeletal body mass while feeding the surgical wire along the guide passageway.

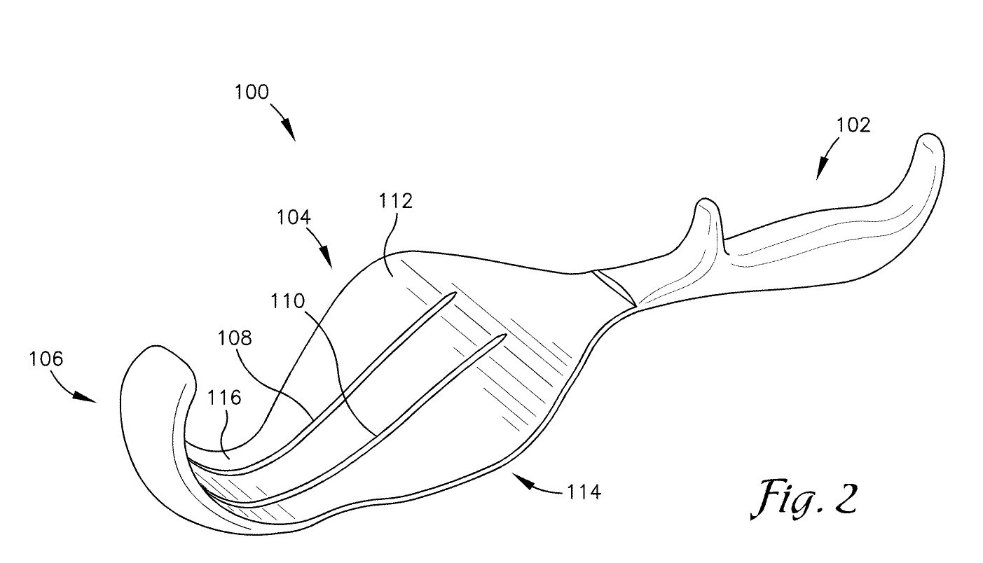

Turning now to FIG. 2, a surgical retractor and wire passer 100 for passing two or more surgical wires around a bone is illustrated in accordance with another embodiment of the invention. The surgical retractor and wire passer 100 broadly comprises an elongated handle 102, a paddle 104, a curved tip 106 and a plurality of guide passageways 108, 110.

The elongated handle 102 extends upward from the paddle 104 and allows a user to grip and apply a leveraging force to the surgical retractor and wire passer 100. The elongated handle 102 may include ergonomic gripping contours, ridges, protrusions, or other geometry for allowing the user to firmly grasp the surgical retractor and wire passer 100. The elongated handle 102 may have a thickness of between 0.5 inches and 2 inches and preferably between 0.5 inches and 1 inch. The elongated handle 102 may have a thickness greater than at least 1.5, preferably at least 2, and more preferably at least 3 times the thickness of the paddle 104.

The paddle 104 is a thin and wide plate for holding non-skeletal body mass away from an operating area and has opposing front and back surfaces 112, 114 and opposing proximal and distal ends. The paddle 104 extends downward from the elongated handle 102 at the proximal end of the paddle 104 and may be angled relative to the elongated handle 102. More specifically, the paddle 104 may be angled at its proximal end or may be angled between its proximal and distal ends. The angle may be between 170 degrees from parallel and 90 degrees and preferably between 160 degrees from parallel and 90 degrees. In this way, the paddle 104 may be substantially flat or may have a slight concave curvature or angle such that the back surface 114 can slightly cradle the non-skeletal body mass. The paddle 104 is connected to or transitions to the curved tip 106 at the distal end of the paddle 104. The paddle 104 may have a thickness of between 0.125 inches and 0.5 inches, preferably between 0.125 inches and 0.4 inches, and more preferably between 0.125 inches and 0.25 inches. The paddle 104 may have a width of between 2 inches and 5 inches and preferably between 3 and 5 inches. The width of the paddle 104 may be at least 2, preferably at least 4, and more preferably at least 6 times the widths of the elongated handle 102 and the curved tip 106.

The curved tip 106 extends from the distal end of the paddle 104 and includes a concave engagement surface 116 for engaging the bone. The concave engagement surface 116 may have a curvature arc of at least 20 degrees, preferably at least 60 degrees, and more preferably at least 90 degrees. The curvature arc may also be at most 180 degrees, preferably at most 160 degrees, and more preferably at most 140 degrees. The concave engagement surface 116 may have a radius of curvature of between 0.25 inches and 2 inches, between 0.5 inches and 4 inches, between 0.75 inches and 3 inches, or between 1 inch and 2 inches for engaging bones of different sizes.

The guide passageways 108, 110 each guide one of the surgical wires at least partially around the bone and may each be a closed-wall, open-ended channel; tube; groove; or other suitable geometry. The guide passageways 108, 110 may extend only along the curved tip 106 or may extend along at least a portion of the paddle 104. The guide passageways 108, 110 may have a diameter or width of at least 0.125 inches and preferably at least 0.25 inches for receiving surgical wires of different sizes. The guide passageways 108, 110 may be spaced between 0.5 inches and 4 inches from each other, preferably between 1 inches and 3 inches from each other, and more preferably between 1 inch and 2 inches from each other. Alternatively, the distances between the guide passageways 108, 110 may be adjustable.

Use of the surgical retractor and wire passer 100 will now be described in more detail. First a user may grasp the elongated handle 12 and insert the curved tip 106 into an operating area near a bone that needs to be braced or reinforced. The concave engagement surface 116 of the curved tip 106 may then be positioned against the bone such that the curved tip 106 cradles the bone. The surgical retractor and wire passer 100 may need to be angled forward briefly to allow the ends of the curved tip 106 to clear the bone.

The back surface 114 of the paddle 104 may then be pushed or pulled against the non-skeletal body mass with the curved tip 106 anchored against the bone so as to hold the non-skeletal body mass away from the operating area. The surgical retractor and wire passer 100 will thus generate leverage, making the non-skeletal body mass easier to hold away from the operating area.

The first surgical wire may then be inserted into the first guide passageway 108 such that the first surgical wire is oriented generally perpendicular to and offset from the bone. The first surgical wire may then be pushed along the first guide passageway 108 towards the end of the curved tip 106 so that the first guide passageway 108 curls the end of the first surgical wire at least partially around the bone. The end of the first surgical wire may then be received on the other side of the bone and the first surgical wire may then be clipped near the paddle 104 between 2 inches and 5 inches from the end of the first surgical wire to form a first wire piece. The first wire piece may then be secured to a brace or other reinforcement near both ends of the first wire piece.

The second surgical wire may then be inserted into the second guide passageway 110 such that the second surgical wire is oriented generally perpendicular to and offset from the bone and spaced from the first wire piece. The second surgical wire may then be pushed along the second guide passageway 110 towards the end of the curved tip 106 so that the second guide passageway 110 curls the end of the second surgical wire at least partially around the bone. The end of the second surgical wire may then be received on the other side of the bone and the second surgical wire may be clipped near the paddle 104 between 2 inches and 5 inches from the end of the second surgical wire to form a second wire piece. The second wire piece may then be secured to the brace or other reinforcement near the ends of the second wire piece. In this way, two wire pieces may be quickly secured to the bone at a predetermined distance from each other. It will be understood that the first and second wires may be inserted around the bone at substantially the same time.

The surgical retractor and wire passer 100 may then be disengaged from the wire pieces (i.e., the wire pieces may be removed from the guide passageways 108, 110). The surgical retractor and wire passer 100 may then be moved laterally (axially along the bone) for passing additional surgical wires around the bone. This may be repeated until the brace is completely secured to the bone.

The curved tip 106 may then be removed from the bone. In doing so, the paddle 104 may release tension on the non-skeletal body mass as the paddle 104 is angled forward. Alternatively, the user may also retain pressure against the non-skeletal body mass via the paddle 104 while disengaging the curved tip 106 from the bone.

The above-described surgical retractor and wire passer 100 provides several advantages over conventional devices. For example, the surgical retractor and wire passer 100 holds non-skeletal body mass away from the operating area while simultaneously guiding the surgical wire around the bone. This eliminates the need for additional tools in order to complete this task. Fewer tools eliminates the reliance on an assistant and/or puts less of a burden on the user, which reduces the cost and time requirement of the procedure. Fewer tools also results in a less cluttered operating area, which makes the surgery easier to perform. A user can easily engage the handle with one hand to move and hold back the non-skeletal body mass while feeding the surgical wires along the guide passageways 108, 110. The two guide passageways 108, 110 allow two surgical wires to be attached to the bone at a predetermined distance from each other for every positioning of the surgical retractor and wire passer 100, which improves accuracy and efficiency of the surgery. It will be understood that additional guide passageways could be used without deviating from the scope of the invention.

Although the invention has been described with reference to the exemplary embodiments illustrated in the attached drawings, it is noted that equivalents may be employed and substitutions made herein without departing from the scope of the invention as recited in the claims.

Having thus described various embodiments of the invention, what is claimed as new and desired to be protected by Letters Patent includes the following:

1. A surgical retractor and wire passer for exposing an operating area and guiding a surgical wire at least partially around a bone, the surgical retractor and wire passer comprising:
an elongated handle;
a paddle having opposing proximal and distal ends, the paddle extending from the elongated handle at the proximal end of the paddle, the paddle being thinner and wider than the elongated handle for holding non-skeletal body mass away from the operating area;
a curved tip extending from the distal end of the paddle, the curved tip presenting a concave engagement surface for engaging the bone and forming a fulcrum point against the bone such that a rearward force applied at the handle creates leverage at the paddle for holding the non-skeletal body mass away from the operating area; and
a guide passageway extending along the concave engagement surface of the curved tip, the guide passageway being configured to receive the surgical wire and guide the surgical wire at least partially around the bone when the concave engagement surface of the curved tip is engaging the bone.

2. The surgical retractor and wire passer of claim 1, wherein the guide passageway extends along at least a portion of the paddle.

3. The surgical retractor and wire passer of claim 1, further comprising a second guide passageway spaced from and parallel to the guide passageway for receiving and guiding a second surgical wire at least partially around the bone when the concave engagement surface of the curved tip is engaging the bone.

4. The surgical retractor and wire passer of claim 3, wherein a distance between the first and second guide passageways is between approximately 0.5 inches and approximately 2 inches.

5. The surgical retractor and wire passer of claim 1, wherein the paddle is at least twice as wide as the handle.

6. The surgical retractor and wire passer of claim 1, wherein the paddle is at least twice as wide as the curved tip.

7. The surgical retractor and wire passer of claim 1, wherein the handle is at least 1.5 times as thick as the paddle.

8. The surgical retractor and wire passer of claim 1, wherein a curvature arc of the concave engagement surface is at least twenty degrees.

9. The surgical retractor and wire passer of claim 1, wherein a radius of curvature of the concave engagement surface is between approximately 0.25 inches and approximately 2 inches.

10. The surgical retractor and wire passer of claim 1, wherein the paddle has a thickness of less than 0.25 inches.

11. The surgical retractor and wire passer of claim 1, wherein the curved tip and the paddle have substantially equal thickness.

12. The surgical retractor and wire passer of claim 1, wherein at least a portion of the paddle is angled relative to the handle.

13. The surgical retractor and wire passer of claim 12, wherein the paddle is bent between its proximal and distal ends.

14. The surgical retractor and wire passer of claim 12, wherein the angle is between 160 degrees and 90 degrees.

15. A method of exposing an operating area and guiding a surgical wire at least partially around a bone, the method comprising the steps of:
a) engaging a curved tip of a surgical retractor against a bone;
b) applying a force to a handle of the surgical retractor while the tip is engaged with the bone to thereby cause a widened paddle of the surgical retractor to hold non-skeletal body mass away from the operating area; and
c) while the curved tip is engaging the bone and the paddle is holding back the non-skeletal body mass, passing a surgical wire around the bone by extending the surgical wire through a guide passageway between the curved tip and the bone.

16. The method of claim 15, further comprising removing the surgical wire from the guide passageway.

17. The method of claim 15, further comprising passing a second surgical wire around the bone by extending the second surgical wire through a second guide passageway between the second curved tip and the bone such that the second surgical wire is spaced form the first surgical wire.

18. The method of claim 17, further comprising removing the second surgical wire from the second guide passageway.

19. The method of claim 17, wherein the first surgical wire and second surgical wire are passed around the bone at substantially the same time.

20. A method of exposing an operating area and guiding two surgical wires at least partially around a bone, the method comprising the steps of:
a) engaging a curved tip of a surgical retractor against a bone;
b) applying a force to a handle of the surgical retractor while the tip is engaged with the bone to thereby cause a widened paddle of the surgical retractor to hold non-skeletal body mass away from the operating area; and
c) while the curved tip is engaging the bone and the paddle is holding back the non-skeletal body mass, passing a first surgical wire around the bone by extending the first surgical wire through a first guide passageway between the first curved tip and the bone and passing a second surgical wire around the bone by extending the second surgical wire through a second guide passageway between the second curved tip and the bone.
